(19) United States
(12) Patent Application Publication
(10) Pub. No.: US 2002/0177757 A1
(43) Pub. Date: Nov. 28, 2002

(54) Systems and methods to facilitate an exchange of information associated with medical care provided to a patient

(75) Inventor: Catherine Britton, Dover, PA (US)

Correspondence Address: Siemens Corporation, Attn: Elsa Keller, Legal Administrator, Intellectual Property Department, 186 Wood Avenue South, Iselin, NJ, 08830, US

(73) Assignee: SIEMENS MEDICAL SYSTEMS, INC.

(21) Appl. No.: 09/864,044

(22) Filed: May 22, 2001

(51) Int. Cl. A61B 5/00

(52) U.S. Cl. 600/300

(57) ABSTRACT

Systems and methods are provided to facilitate an exchange of information associated with a patient's medical care. According to one embodiment, medical care information is exchanged with a medical care information device, such as the LANTIS® Oncology Management System. Patient information is then exchanged with a remote patient device via a communication network, the patient information being associated with the medical care information. For example, the patient may exchange scheduling information, journal information, information associated with another patient or a support group, educational information, and/or medical instructions via a Web site.

### BACKGROUND OF THE INVENTION

1. Field of the Invention

The present invention relates to patients and medical care providers. In particular, the present invention relates to systems and methods to facilitate an exchange of information associated with a patient's medical care.

2. Description of the Related Art

An important aspect of medical care involves an exchange of information between a patient and a medical care provider. For example, the medical care provider may need to provide scheduling information to the patient (e.g., dates and times associated with oncology treatment procedures). Moreover, the patient may also want to provide information to the medical care provider. For example, the patient may want to provide medical care information (e.g., how he or she has been feeling) or adjust his or her treatment schedule.

Typically, the medical care provider manually provides information to the patient. For example, the medical care provider can verbally give scheduling information to the patient. The medical care provider might also give the patient a hand-written (or computer-printed) list of scheduled appointments. The patient then manually transfers the information, such as by writing scheduling information on his or her calendar or entering the information into a Personal Digital Assistant (PDA). It is also known that a patient can manually store information via a Web site. For example, a My Calendar feature offered by the oncology.com^{SM} Web site lets a patient manually enter and store scheduling information associated with his or her medical care.

There are a number of disadvantages, however, when information is exchanged between a patient and a medical care provider in these ways. For example, the patient may find it inconvenient to manually transfer the information. Moreover, the patient may forget to transfer the information or introduce errors when transferring the information (e.g., by entering the wrong date into his or her PDA). These disadvantages may be especially significant with respect to medical care that requires ongoing treatments, such as oncology related medical care.

In addition, known approaches do not facilitate a transfer of information from the patient to the medical care provider. For example, a patient who wants to adjust his or her treatment schedule may need to manually indicate the adjustment to the medical care provider (e.g., via a telephone call) and then update the information stored in his or her PDA accordingly.

Even if a patient could directly access and modify information used by a medical care provider, such as a treatment schedule database, several problems may arise. For example, the patient may inadvertently introduce scheduling errors, such as by scheduling an appointment when a particular doctor or piece of medical equipment is not available. Similarly, the patient may unknowingly adjust his or her schedule such that a follow-up exam becomes scheduled before an associated medical procedure.

A medical care provider may also need to provide a medical instruction to a patient. For example, the medical care provider may need to tell a patient that he or she must take a particular medication twenty-four hours before a scheduled appointment. Typically a medical care provider manually provides such a medical instruction to a patient (e.g., via a telephone call). However, a patient may forget to follow such an instruction.

A patient may also be interested in receiving educational material from a medical care provider. For example, a patient who has been diagnosed with breast cancer may perform research (e.g., via the Web) and find several recently published articles. The patient, however, may be unsure if he or she should trust the information found in those articles. In this case, the patient typically asks his or her medical care provider for an opinion. Such an approach is inefficient because the medical care provider may receive many such requests (from a large number of patients). Moreover, patients with different medical conditions may need to receive different types of educational material.

### SUMMARY OF THE INVENTION

To alleviate problems inherent in the prior art, the present invention introduces systems and methods to facilitate an exchange of information associated with a patient's medical care.

According to one embodiment, medical care information is exchanged with a medical care information device, the medical care information device being in communication with a medical care provider device associated with a medical care provider. In addition, patient information is exchanged with a remote patient device via a communication network, the patient information being associated with the medical care information.

Another embodiment is directed to a computer-implemented method for facilitating an exchange of information associated with medical care provided to a patient by a medical care provider. According to this embodiment, treatment scheduling information associated with the patient is retrieved from a medical care information database, the medical care information database also being accessed by a medical care provider device associated with the medical care provider. An image of the patient is also retrieved and transmitted to the patient, along with the treatment scheduling information, via a Web site. An indication associated with the treatment scheduling information is received from the patient via the Web site and converted into an electronic mail message. It is then arranged for the electronic mail message to be transmitted to the medical care provider device.

According to another embodiment, patient information is exchanged at a patient device via a communication network, the patient information being based on medical care information stored at a medical care information device accessed by a medical care provider. In addition, an indication associated with the patient information is transmitted from the patient device.

According to another embodiment, scheduling information is received at a patient device via a communication network, the scheduling information being based on information stored at a medical care information device accessed by the medical care provider. The patient device then transmits a request to adjust the scheduling information.

According to still another embodiment, medical care information is exchanged with a medical care information device, the medical care information device being in communication with a controller that that transmits patient information to a remote patient device based on the medical care information. In addition, an indication associated with the patient information is received.

According to yet another embodiment, medical care information is exchanged with a medical care information device via a proprietary communication network. In addition, patient information is exchanged with a remote patient device via a public communication network, the patient information being associated with the medical care information.

One embodiment of the present invention comprises: means for exchanging medical care information with a medical care information device, the medical care information device being in communication with a medical care provider device associated with a medical care provider; and means for exchanging patient information with a remote patient device via a communication network, the patient information being associated with the medical care information.

Another embodiment comprises: means for retrieving treatment scheduling information associated with a patient from a medical care information database, the medical care information database also being accessed by a medical care provider device associated with a medical care provider; means for retrieving an image of the patient; means for transmitting to the patient the treatment scheduling information and the image of the patient via a Web site; means for receiving from the patient an indication associated with the treatment scheduling information via the Web site; means for converting the indication into an electronic mail message; and means for arranging for the electronic mail message to be transmitted to the medical care provider device.

Another embodiment comprises: means for exchanging patient information at a patient device via a communication network, the patient information being based on medical care information stored at a medical care information device accessed by a medical care provider; and means for transmitting an indication associated with the patient information.

Another embodiment comprises: means for receiving scheduling information at a patient device via a communication network, the scheduling information being based on information stored at a medical care information device accessed by a medical care provider; and means for transmitting a request to adjust the scheduling information.

Still another embodiment comprises: means for exchanging medical care information with a medical care information device, the medical care information device being in communication with a controller that that transmits patient information to a remote patient device based on the medical care information; and means for receiving an indication associated with the patient information.

Yet another embodiment comprises: means for exchanging medical care information with a medical care information device via a proprietary communication network; and means for exchanging patient information with a remote patient device via a public communication network, the patient information being associated with the medical care information.

With these and other advantages and features of the invention that will become hereinafter apparent, the invention may be more clearly understood by reference to the following detailed description of the invention, the appended claims, and the attached drawings.

### BRIEF DESCRIPTION OF THE DRAWINGS

FIG. 1 is a block diagram overview of a medical care information system according to an embodiment of the present invention.

FIG. 2 is a flow chart of a method for facilitating an exchange of information according to an embodiment of the present invention.

FIG. 3 illustrates patient information displays according to an embodiment of the present invention.

FIG. 4 is a block diagram of a patient device according to an embodiment of the present invention.

FIGS. 5 through 7 illustrate patient devices displaying medical care information according to some embodiments of the present invention.

FIG. 8 is a block diagram of a controller according to an embodiment of the present invention.

FIG. 9 is a tabular representation of a portion of a patient database according to an embodiment of the present invention.

FIG. 10 is a tabular representation of a portion of a scheduling database according to an embodiment of the present invention.

FIG. 11 is a tabular representation of a portion of a supplemental information database according to an embodiment of the present invention.

FIG. 12 is a flow chart of a computer-implemented method for facilitating an exchange of information at a controller according to an embodiment of the present invention.

FIG. 13 is a flow chart of a method for facilitating an exchange of information at a medical care provider device according to an embodiment of the present invention.

### DETAILED DESCRIPTION

The present invention is directed to systems and methods that facilitate an exchange of information associated with “medical care” provided to a patient. As used herein, the phrase “medical care” may refer to, for example, any medical evaluation, diagnosis, or treatment associated with a patient. Some examples of medical care include oncology care, dental care, and psychiatric care.

As used herein, the phrase “medical care information” may refer to any information exchanged with a medical care provider that is associated with medical care provided to a patient. Medical care information may include, for example, medical diagnosis information, scheduling information, journal information, information associated with another patient or a support group, educational information, medical instructions, and/or indications received from a patient (e.g., a request to adjust scheduling information).

As used herein, the phrase “patient information” may refer to any information exchanged with a patient that is associated with the patient's medical care. Patient information may refer to, for example, scheduling information, journal information, information associated with another patient or a support group, educational information, medical instructions, and/or indications generated by the patient (e.g., a request to adjust scheduling information). Note that patient information may, for example, comprise a subset of associated medical care information accessed by a medical care provider.

Medical Care Information System Overview

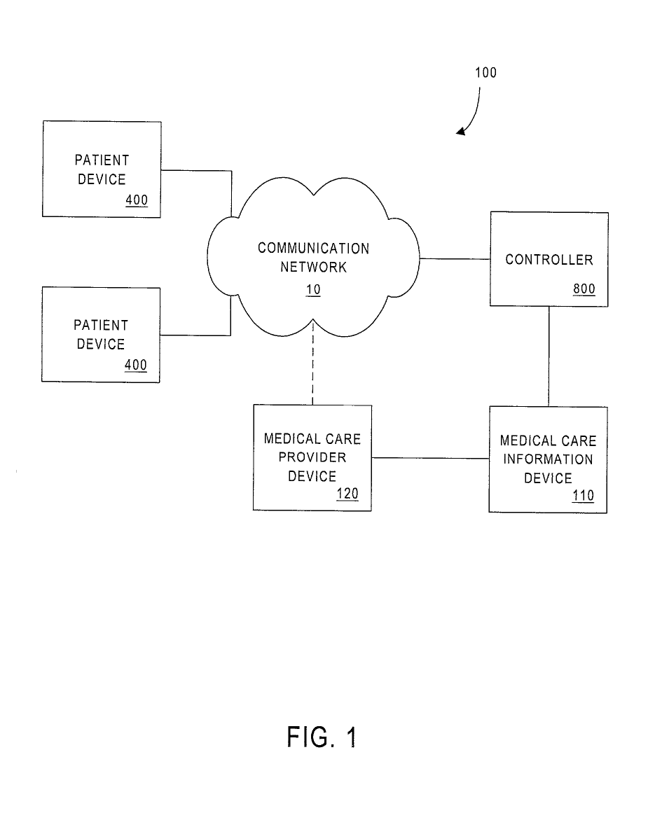

Turning now in detail to the drawings, FIG. 1 is a block diagram of a medical care information system 100 according to one embodiment of the present invention. The medical care information system 100 includes a controller 800 in communication with a number of remote patient devices 400. As used herein, devices (such as the patient devices 400 and the controller 800) may communicate, for example, via a communication network 10, such as a Local Area Network (LAN), a Metropolitan Area Network (MAN), a Wide Area Network (WAN), a proprietary network, a Public Switched Telephone Network (PSTN), a Wireless Application Protocol (WAP) network, a cable television network, or an Internet Protocol (IP) network such as the Internet, an intranet or an extranet. Moreover, as used herein, communications include those enabled by wired or wireless technology. Note that although a single controller 800 is shown in FIG. 1, any number of controllers 800 may be included in the medical care information system 100. Similarly, any number of the other devices described herein may be included in the medical care information system 100 according to embodiments of the present invention.

In one embodiment, a patient device 400 communicates with a remote, Web-based controller 800 (e.g., a server) via the Internet. Although some embodiments are described with respect to information exchanged via a Web site, according to other embodiments information is instead exchanged, for example, via: a telephone, an Interactive Voice Response Unit (IVRU), electronic mail, a WEBTV® interface, a cable network interface, and/or a wireless communication system.

The patient device 400 and the controller 800 may be any devices capable of performing various functions described herein. The patient device 400 may be, for example: a Personal Computer (PC), a portable computing device such as a PDA, a wired or wireless telephone, a one-way or two-way pager, a kiosk (e.g., a kiosk located at a hospital), an interactive television device, or any other appropriate storage and/or communication device.

Note that the devices shown in FIG. 1 need not be in constant communication. For example, the patient device 400 may only communicate with the controller 800 via the Internet when appropriate (e.g., when attached to a “docking” station or “cradle” coupled to the patient's PC). The patient device 400 may also communicate with the controller 800 via an infrared device when near a medical care information kiosk (e.g., a kiosk located at a medical care clinic).

The controller 800 is also in communication with a medical care information device 110 that stores information associated with medical care provided to patients. The medical care information device 110 may be associated with, for example, the LANTIS® Oncology Management System developed by SIEMENS MEDICAL SYSTEMS®. The LANTIS® Oncology Management System is a suite of oncology information management modules that can securely access a comprehensive, integrated Btrieve/Pervasive® Structured Query Language (SQL) database containing electronic medical records, scheduling information, budget and cost information, chemotherapy charting, outcome management, clinical imaging, and transcription information.

According to one embodiment, the medical care information device 110 may be associated with the controller 800 (e.g., both may be operated by a hospital). According to another embodiment, the controller 800 and the medical care information device 110 are operated by different parties.

The medical care information device 110 also communicates with one or more medical care provider devices 120. A medical care provider device 120 may be, for example, a PC or a workstation used by doctors and nurses to access medical care information associated with the LANTIS® Oncology Management System (e.g., via a C++ application being executed at a medical care provider device 120).

According to the present invention, a patient uses a patient device 400 to exchange information associated with his or her medical care via a controller 800. For example, a patient may view his or her medical treatment schedule via the patient device 400. The patient device 400 may also be used to provide or generate patient inputs. For example, a patient may use a keyboard or mouse coupled to his or her PC to request a change to his or her treatment schedule.

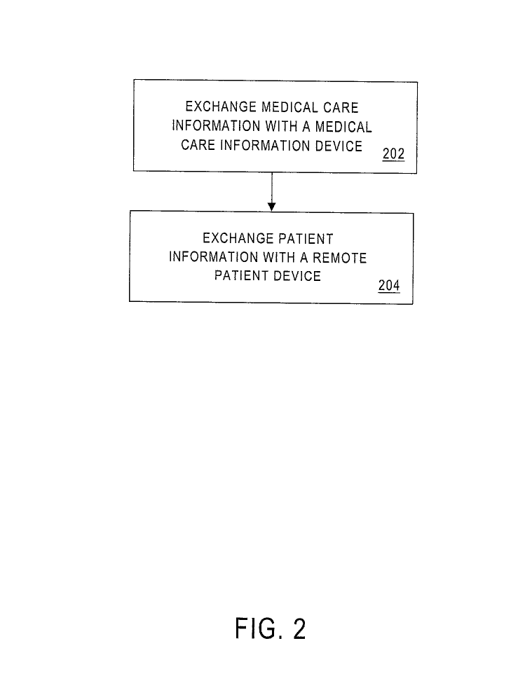

FIG. 2 is a flow chart of a method for facilitating an exchange of information according to an embodiment of the present invention. The flow charts in FIG. 2 and the other figures described herein do not imply a fixed order to the steps, and embodiments of the present invention can be practiced in any order that is practicable. Moreover, the methods may be performed by any of the devices described herein. The method shown in FIG. 2 may be performed, for example, by the controller 800.

At 202, medical care information is exchanged with the medical care information device 110. For example, the controller 800 may retrieve scheduling information from a LANTIS® Oncology Management System associated with the medical care provider, a hospital, a medical clinic, and/or a doctor. The retrieved scheduling information may be associated with, for example, a medical examination, a treatment, or procedure and may indicate, for example, the medical care provider and treatment, a medical care category (e.g., “radiology”), an appointment date and time, and/or an appointment duration.

According to one embodiment of the present invention, the controller 800 communicates with a number of different medical care information devices 110 (e.g., associated with a number of different hospitals or medical clinics). In this case, the medical care information that is retrieved at 202 may be associated with a number of different medical care information devices 110 and/or medical care providers.

At 204, patient information a remote patient device 400 via a communication network 10. For example, the controller 800 may transmit scheduling information to a patient's PC via the Internet.

According to one embodiment, the controller 800 also determines if the scheduling information includes any updates. For example, the controller 800 may compare the scheduling information with an older version to determine if any appointments have been changed by the medical care provider. If so, the controller 800 may transmit an indication of the update to the patient device 400 (e.g., by displaying “Please note that your February 17th Follow-Up Exam has been moved to February 25th”).

The controller 800 may also receive an indication associated with the scheduling information from the patient device 400. For example, the patient may access a Web site associated with the controller 800 to request a change to a scheduled appointment (or to confirm an existing appointment). In this case, the controller 800 can arrange for the indication to be transmitted to the medical care provider device 120. For example, the controller 800 may convert a Hypertext Markup Language (HTML) indication received via the Web site into an electronic mail message and transmit the electronic mail message to the medical care provider device 120 (e.g., via the medical care information device 110 or the communication network 10).

According to another embodiment, the controller 800 also retrieves supplemental information associated with the patient (e.g., from a local database or from the medical care information device 110) and transmits the supplemental information to the patient device 400. The supplemental information may include, for example, the patient's name, medical care information associated with the patient (e.g., a recent medical article associated with a particular medical condition), third party information (e.g., contact information for a local support group), information associated with a medical care provider (e.g., directions to a medical clinic), reminder information (e.g., associated with a medication), and/or information associated with at least one other patient (e.g., an indication that a neighbor with a similar medical condition is interested in contacting the patient). The supplemental information may also be an image of the patient, which can help re-assure the patient that the correct information is being displayed (e.g., via a Web site).

It is important to note that, according to one embodiment, the controller 800 exchanges information with the medical care information device 110 via a first communication network and the patient device 400 via a second communication network. In particular, the controller 800 may access sensitive medical care information (e.g., a patient's electronic medical record) via a proprietary network (e.g., a private and/or secure network). The controller 800 may use that information, for example, to match a patient with another patient who (i) has a similar medical condition and (ii) has indicated that he or she is interested in sharing information (e.g., an electronic mail address) with other patients. The controller 800 may then send less sensitive information (e.g., scheduling information or another patient's electronic mail address) via a public network (e.g., the Internet). In this way, the confidential nature of the patient's medical care information may be protected.

According to another embodiment, the controller 800 also receives supplemental patient information from the patient device 400. The controller 800 may then store the information or transmit the information to the medical care information device 110 (or the medical care provider device 120). The supplemental patient information may comprise, for example, text, audio, and/or image information associated with a patient's journal (e.g., in which a patient describes how he or she is feeling). The supplemental patient information may also be associated with, for example, general scheduling information (e.g., “please do not schedule future appointments before 10:00 AM”) and/or medical care information (e.g., a question for the medical care provider regarding a side effect being caused by a medication).

According to one embodiment, the controller 800 locally stores the supplemental patient information until an indication is received from the patient device 400 that the information should be provided to the medical care provider. For example, the controller 800 may locally store the patient's journal until the patient indicates that one or more journal entries should be transmitted to the medical care provider.

Many of the embodiments described above are associated with scheduling information transmitted from the controller 800 to the patient device 400. According to another embodiment, the patient information exchanged at 204 comprises the controller 800 receiving journal information from a patient device 400. For example, a patient may keep a daily journal via a Web site associated with the controller 800 (e.g., including text, audio, and/or image information). In this case, the medical care information exchanged at 202 may include, for example, some or all of the patient's journal entries. For example, the patient may use the Web site to indicate which journal entries should be forwarded to his or her doctor (e.g., via the medical care information device 110).

According to another embodiment, the medical care information exchanged at 202 includes a patient's medical diagnosis information. For example, the controller 800 may determine the patient's medical condition based on information stored at the medical care information device 110. In this case, the patient information exchanged at 204 includes information associated with another patient or information associated with a support group that is transmitted to the patient device 400. For example, the controller 800 may use the medical diagnosis information associated with a first patient to find a second patient who has a similar medical condition. The second patient or a local support group may also be selected based on, for example, address information (e.g., zip codes or telephone area codes).

Another embodiment also involves the controller 800 receiving a patient's medical diagnosis information at 202. In this embodiment, the patient information exchanged at 204 includes educational information that is transmitted to the patient device 400. For example, the controller 800 may retrieve a number of articles (e.g., recently published in medical journals) associated with the patient's medical condition. The educational information transmitted to the patient device 400 may be generated or reviewed by the medical care provider (e.g., to ensure that only accurate educational information is provided to the patient device 400). Individual articles may be reviewed by the medical care provider, or the medical care provider may indicate that all articles associated with a particular author or publication are acceptable (assuming the information is related to the patient's medical condition).

According to another embodiment the medical care information exchanged at 202 comprises the controller 800 receiving one or more medical instructions from the medical care information device 110. For example, a doctor or nurse may store medicals instructions for a patient via the medical care information device 110. In this case, and the patient information exchanged at 204 will include the appropriate medical instructions. For example, the controller 800 may transmit the medical instructions to the patient device 400 when the patient accesses a Web site. According to one embodiment, the patient may also provide an indication to the controller 800 that he or she has complied with the medical instructions (e.g., a patient may check-off medical instructions as he or she complies). The controller 800 may then arrange for these indications to be provided to the medical care provider device 120 (e.g., via the medical care information device). The controller 800 may also arrange for these indications to be provided to the patient device 400 the next time the patient accesses the Web site (e.g., to remind the patient which medical instructions have already been complied with).

### EXAMPLE

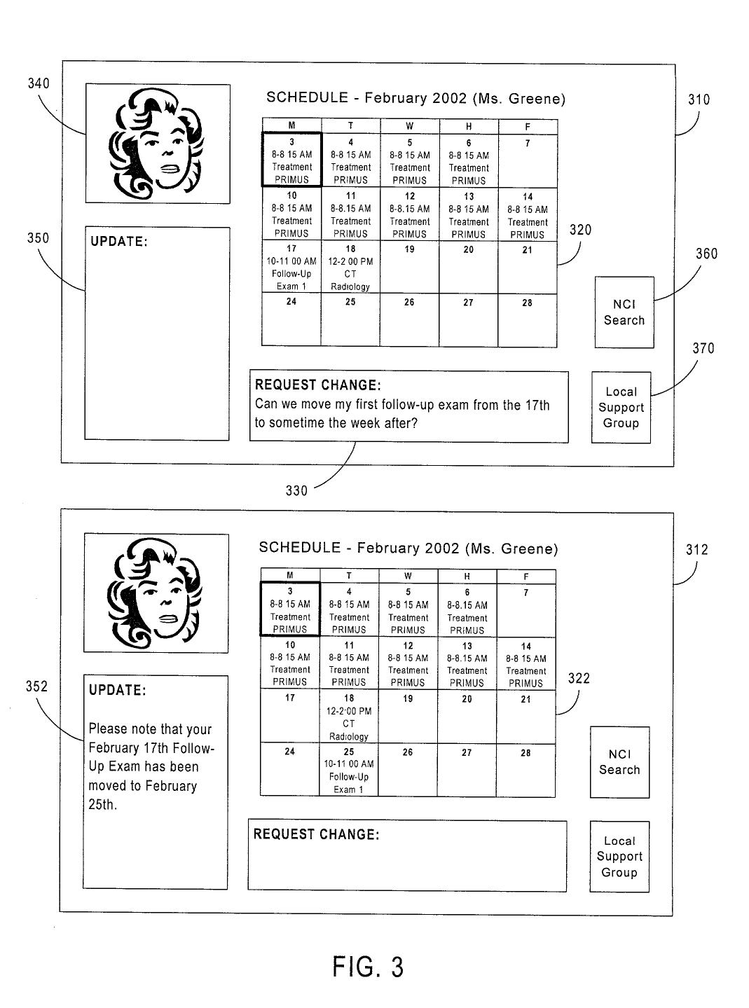

By way of example, consider the first patient display 310 shown in FIG. 3. In this case, the patient display 310 includes scheduling information 320 for Ms. Greene indicating the appointments she has scheduled with a medical care provider in February, 2002. In particular, the scheduling information 320 indicates a date, a time, and a category (e.g., “treatment” or “exam”) for each scheduled appointment. According to one embodiment, different types of appointments may be displayed in different colors (e.g., red indicating treatments and yellow indicating clinical visits). The scheduling information 320 may be displayed, for example, as a list or in a daily, weekly, or monthly format.

The patient display 310 also includes a request change area 330, a patient image area 340 (e.g., displaying a picture of the patient), and an update area 350 (e.g., indicating any schedule updates to the patient). The request change area 330 may be used, for example, by the patient to generate a schedule adjustment request. As shown in FIG. 3, Ms. Greene has asked to move her follow-up exam from February 17th to sometime during the next week. According to an embodiment of the present invention, this requested is received by the controller 800 (e.g., via an HTML message), converted (e.g., into an electronic mail message), and forwarded to a medical care information device 110 (e.g., a LANTIS® Oncology Management System) and/or a medical care provider device 120. The second patient display 312 illustrates adjusted scheduling information 322 and an update area 352 responsive to Ms. Greene's request.

The patient display 310 may also let a patient request information associated with a third party (e.g., other than the patient and the medical care provider). For example, the National Cancer Institute (NCI) search request 360 may find recently published articles or other educational information associated with Ms. Greene's medical condition. The local support group request 370 may display a list of local support groups associated with her medical condition (e.g., based on her home address) or may arrange for her to contact other patients with a similar medical condition.

Patient Device

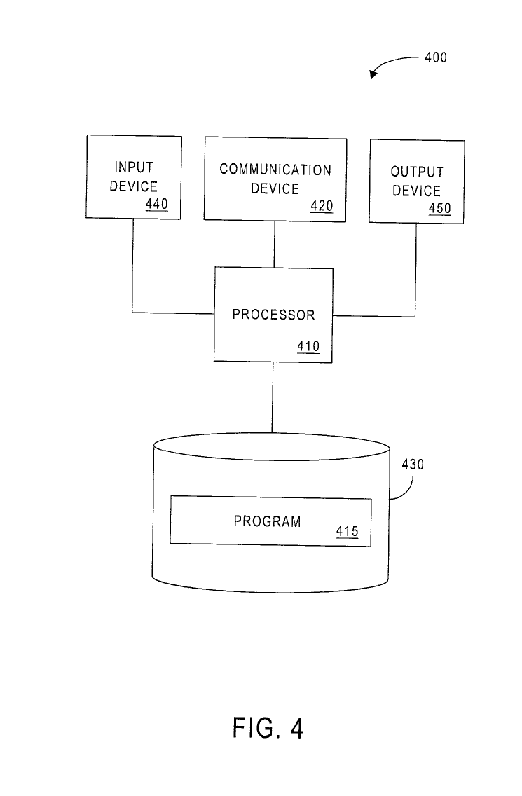

FIG. 4 illustrates a patient device 400 that is descriptive of the device shown in FIG. 1 according to an embodiment of the present invention. The patient device 400 comprises a processor 410, such as one or more INTEL® Pentium® processors, coupled to a communication device 420 configured to communicate via a communication network (not shown in FIG. 4). The communication device 420 may be used to communicate, for example, with the controller 800 and/or a medical care provider device 120.

The processor 410 is also in communication with an input device 440. The input device 440 may comprise, for example, a keyboard, a mouse or other pointing device, a microphone, a knob or a switch (including an electronic representation of a knob or a switch), an infrared port, a docking station, and/or a touch screen. Such an input device 440 may be used, for example, to provide patient inputs (e.g., to request an adjustment to scheduling information).

The processor 410 is also in communication with an output device 450. The output device 440 may comprise, for example, a display (e.g., a computer monitor), a speaker, and/or a printer. The output device 450 may be used, for example, to provide medical care information to a patient (e.g., by displaying scheduling information to the patient).

The processor 410 is also in communication with a storage device 430. The storage device 430 may comprise any appropriate information storage device, including combinations of magnetic storage devices (e.g., magnetic tape and hard disk drives), optical storage devices, and/or semiconductor memory devices such as Random Access Memory (RAM) devices and Read Only Memory (ROM) devices.

The storage device 430 stores a program 415 for controlling the processor 410. The processor 410 performs instructions of the program 415, and thereby operates in accordance with the present invention. For example, the processor 410 may receive scheduling information from the controller 800 and transmit a request to adjust the scheduling information to the controller 800.

As used herein, information may be “received” by or “transmitted” to, for example: (i) the patient device 400 from the controller 800; or (ii) a software application or module within the patient device 400 from another software application, module, or any other source.

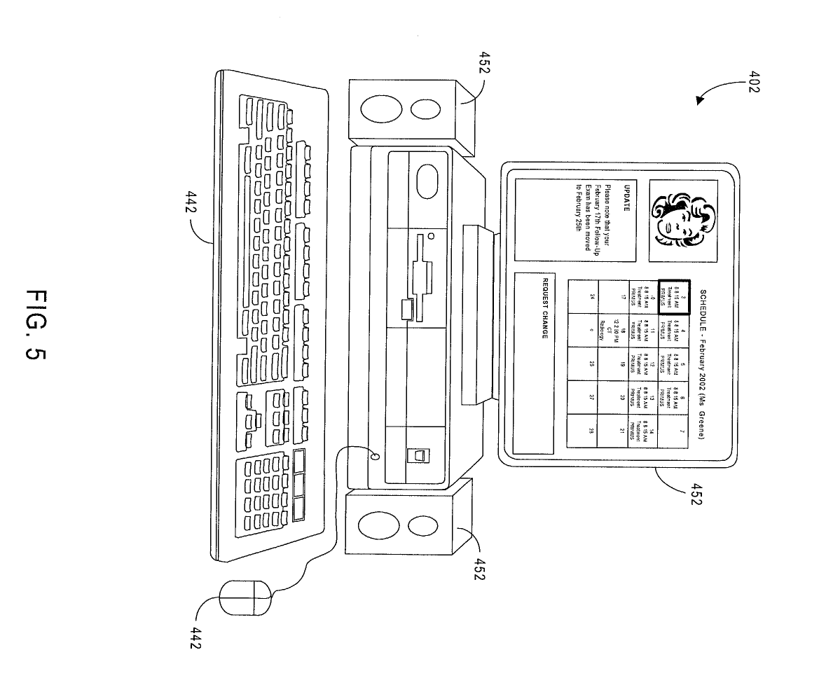

FIG. 5 illustrates a PC 402 displaying medical care information according to an embodiment of the present invention. The PC 402 includes a keyboard and a mouse 442 which can be used by a patient to provide patient inputs (e.g., a request to adjust scheduling information). The PC 402 also includes a computer display and speakers 452 which can be used, for example, to indicate medical care information to the patient.

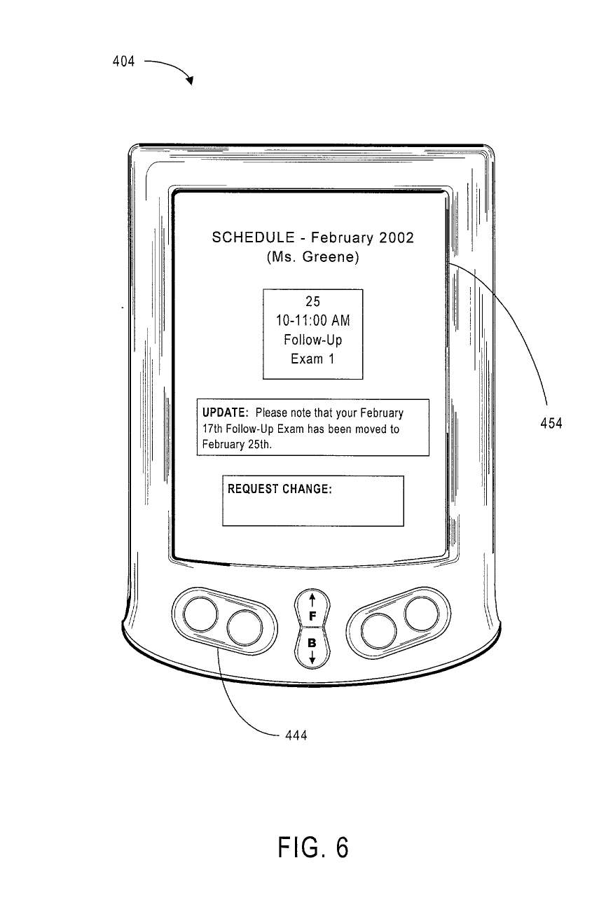

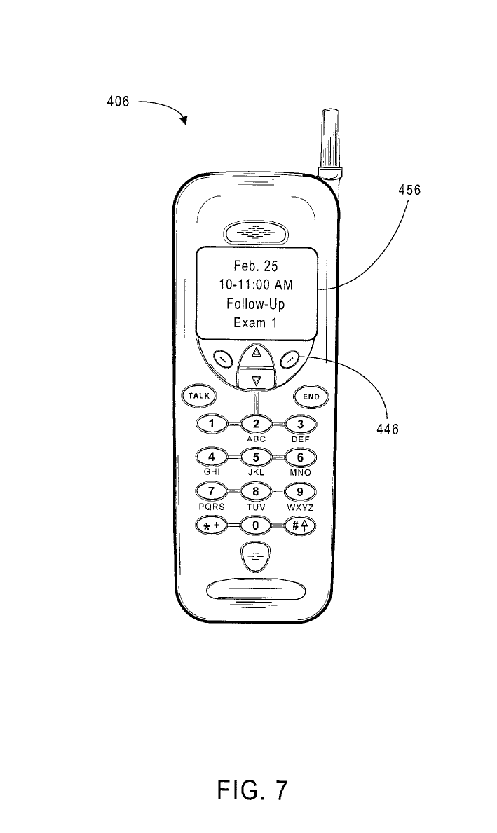

FIG. 6 illustrates a PDA 404 displaying medical care information according to another embodiment of the present invention. The PDA 404 includes an input device 444 and an output device 454 (e.g., a display screen) that may be used by a patient. Similarly, FIG. 7 illustrates a wireless telephone 406 including an input device 446 and an output device 456 displaying medical care information.

Controller

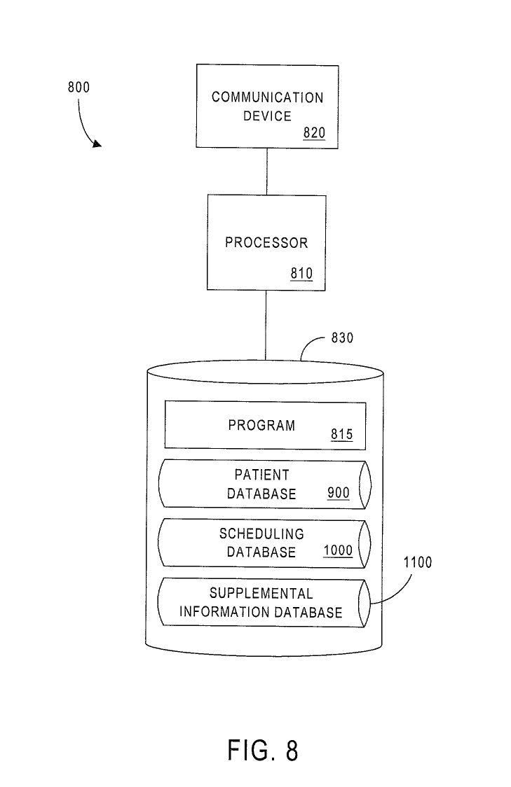

FIG. 8 illustrates a controller 800 that is descriptive of the device shown in FIG. 1 according to an embodiment of the present invention. The controller 800 comprises a processor 810, such as one or more INTEL® Pentium® processors, coupled to a communication device 820 configured to communicate via a communication network (not shown in FIG. 8). The communication device 820 may be used to communicate, for example, with one or more patient devices 400, medical care information devices 110, and medical care provider devices 120.

The processor 810 is also in communication with a storage device 830. The storage device 830 may comprise any appropriate information storage device, including combinations of magnetic storage devices (e.g., magnetic tape and hard disk drives), optical storage devices, and/or semiconductor memory devices such as RAM devices and ROM devices.

The storage device 830 stores a program 815 for controlling the processor 810. The processor 810 performs instructions of the program 815, and thereby operates in accordance with the present invention. For example, the processor 810 may retrieve from a medical care information device 110 scheduling information associated with a patient and transmit the scheduling information to a patient device 400.

According to another embodiment, the processor 810 retrieves treatment scheduling information associated with the patient from a medical care information database along with an image of the patient. The processor 810 then transmits the treatment scheduling information and the image of the patient to a patient device 400 via a Web site. The processor 810 may also receive from the patient device 400 an indication associated with the treatment scheduling information via the Web site and convert the indication into an electronic mail message. The processor 810 can then arrange for the electronic mail message to be transmitted to a medical care provider device 120 (e.g., via a medical care information device 110).

The program 815 may be stored in a compressed, uncompiled and/or encrypted format. The program 815 may furthermore include other program elements, such as an operating system, a database management system, and/or device drivers used by the processor 810 to interface with peripheral devices.

As used herein, information may be “received” by or “transmitted” to, for example: (i) the controller 800 from the patient device 400; or (ii) a software application or module within the controller 800 from another software application, module, or any other source.

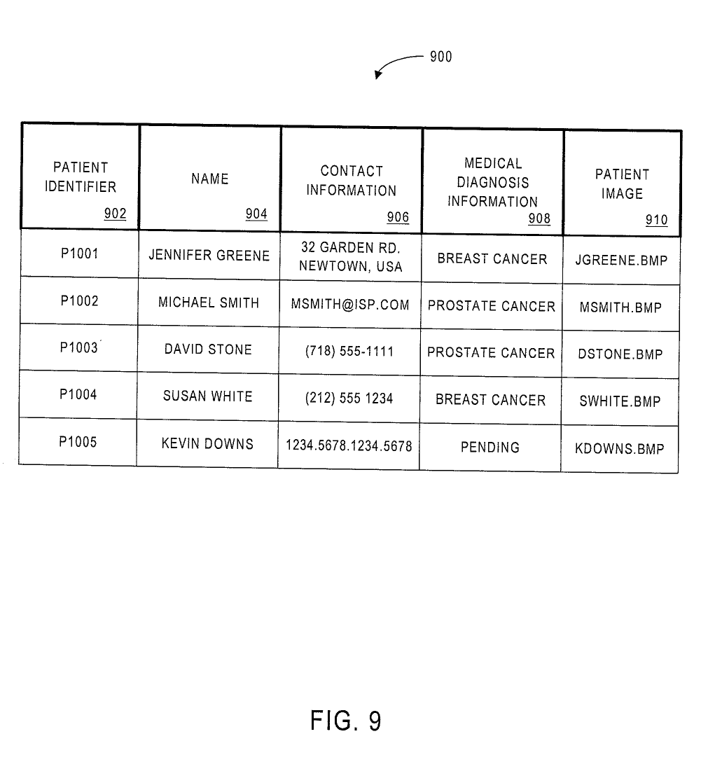

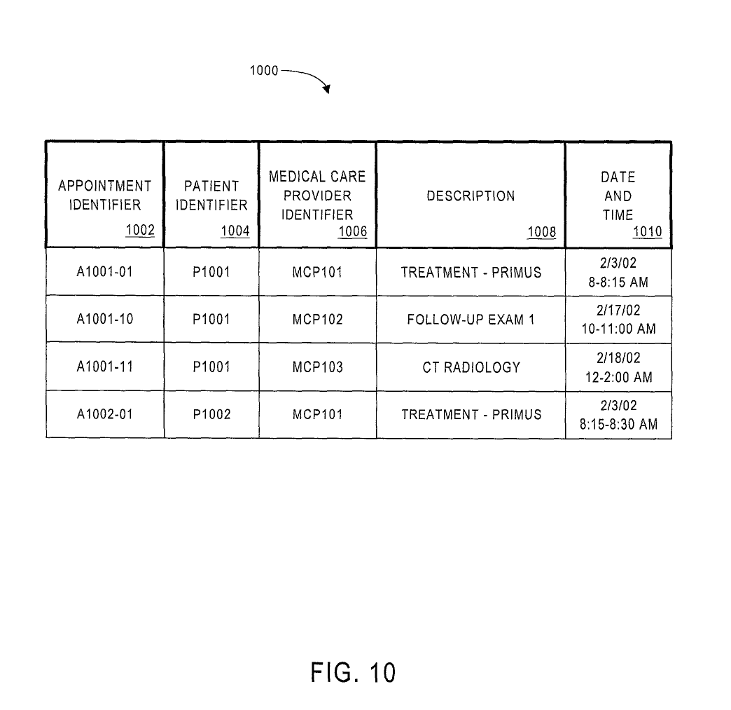

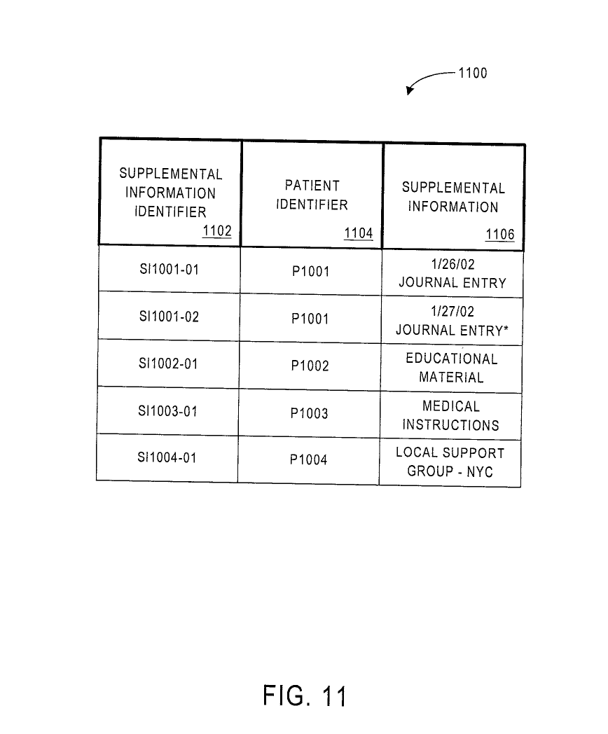

As shown in FIG. 8, the storage device 830 also stores a patient database 900 (described with respect to FIG. 9), a scheduling database 1000 (described with respect to FIG. 10), and a supplemental information database 1100 (described with respect to FIG. 11). Examples of databases that may be used in connection with the controller 800 will now be described in detail with respect to FIGS. 9 through 11. Note that the illustrations and accompanying descriptions of the databases presented herein are exemplary, and any number of other database arrangements could be employed besides those suggested by the figures.

Patient Database

Referring to FIG. 9, a table represents the patient database 900 that may be stored at the controller 800 according to an embodiment of the present invention. The table includes entries identifying patients who may exchange information via the medical care information system 100. The table also defines fields 902, 904, 906, 908, 910 for each of the entries. The fields specify: a patient identifier 902, a name 904, contact information 906, medical diagnosis information 908, and a patient image 910. The information in the patient database 900 may be created and updated, for example, based on information received from patient when he or she registers with the controller 800. The information in the patient database 900 may also be based on, for example, information received from the medical care information device 110 (e.g., associated with a LANTIS® Oncology Management System).

The patient identifier 902 may be, for example, an alphanumeric code associated with a patient who has registered to use the medical care information system 100. The patient identifier 902 may be generated by, for example, the controller 800 or the patient (e.g., when he or she provides a user name and password) and may be based on, or associated with, a patient identifier stored in the medical care information device 110. The patient database 900 also stores the name 904 and contact information 906 (e.g., a postal address, an electronic mail address, an IP address, or a telephone number) associated with each patient.

The medical diagnosis information 908 may indicate, for example, a medical condition associated with the patient. The medical diagnosis information 908 may be used, for example, to let a patient contact another patient having a similar medical condition. For example, as shown in FIG. 9, the controller 800 may arrange for Jennifer Greene to contact Susan White (assuming, of course, that Susan White agreed to be contacted by other patients) because they both have breast cancer.

The patient image 910 may comprise, for example, a bitmap or other type of photographic image of the patient. The patient image 910 may be, for example, transmitted to a remote patient device 400 to reassure the patient that the correct information being provided.

Some of all of the information shown in FIG. 9 instead by stored at the patient device 400 (e.g., as a Web browser “cookie” file).

Scheduling Database

Referring to FIG. 10, a table represents the scheduling database 1000 that may be stored at the controller 800 according to an embodiment of the present invention. The table includes entries associated with medical care appointments. The table also defines fields 1002, 1004, 1006, 1008, 1010 for each of the entries. The fields specify: an appointment identifier 1002, a patient identifier 1004, a medical care provider identifier 1006, a description 1008, and a date and time 1010. The information in the scheduling database 1000 may be created and updated, for example, based on information received from the medical care information device 110 (e.g., associated with a LANTIS® Oncology Management System). That is, the controller 800 may retrieve this information from the medical care information device 110 and use the information to dynamically generate a Web page when a patient accesses a Web site.

The appointment identifier 1002 may be, for example, an alphanumeric code associated with a medical care appointment. The patient identifier 1004 represents a patient associated with the appointment and may be based on, or associated with, the patient identifier 902 stored in the patient database 900. The medical care provider 1006 represents a medical care provider (e.g., a hospital, clinic, doctor, or nurse) associated with the appointment and the description 1008 describes the appointment (e.g., by indicating that the appoint is for “CT Radiology”). The date and time 1010 indicate when the appointment is scheduled.

Supplemental Information Database

Referring to FIG. 11, a table represents the supplemental information database 1100 that may be stored at the controller 800 according to an embodiment of the present invention. The table includes entries identifying supplemental information that may be exchanged via the medical care information system 100. The table also defines fields 1102, 1104, 1106 for each of the entries. The fields specify: a supplemental information identifier 1102, a patient identifier 1104, and supplemental information 1106. The information in the supplemental database 1100 may be created and updated, for example, by the controller 800 based on information received from a patient device 400 and/or the medical care information device 110.

The supplemental identifier 1102 may be, for example, an alphanumeric code associated with supplemental information that has been, or may be, exchanged via the medical care information system 100. The patient identifier 1004 represents a patient associated with the supplemental information and may be based on, or associated with, the patient identifier 902 stored in the patient database 900 and/or the patient identifier 1004 stored in the scheduling database 1000.

The supplemental information 1106 contains the supplemental information and may represent, for example, a patient's journal entry. In this case, the supplemental information 1106 may further indicate whether or not the journal entry should be forwarded to a medical care provider (e.g., as illustrated by a “*” in FIG. 11). The supplemental information 1106 may also comprise educational material, medical instructions, and/or a local support group or another patient associated with a particular medical condition.

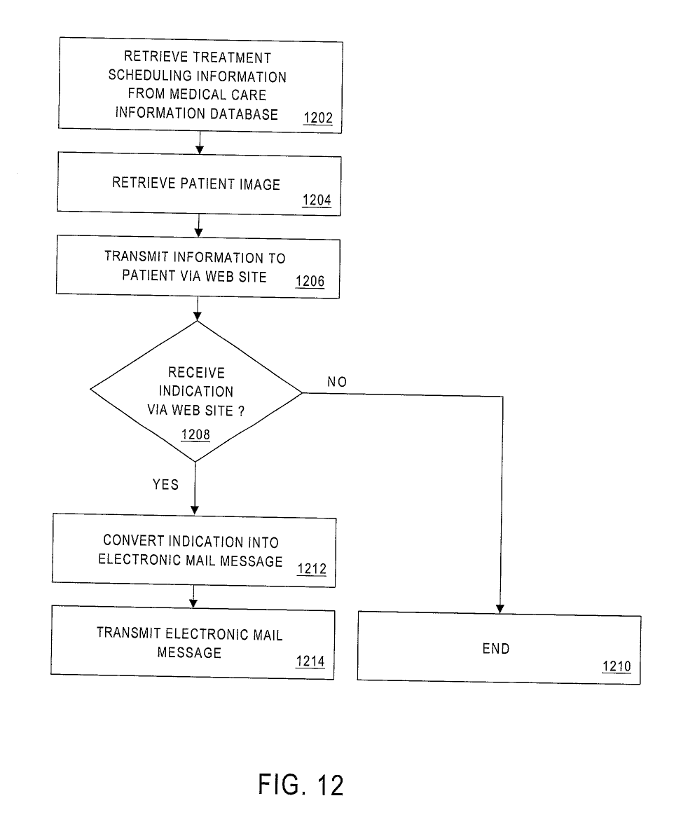

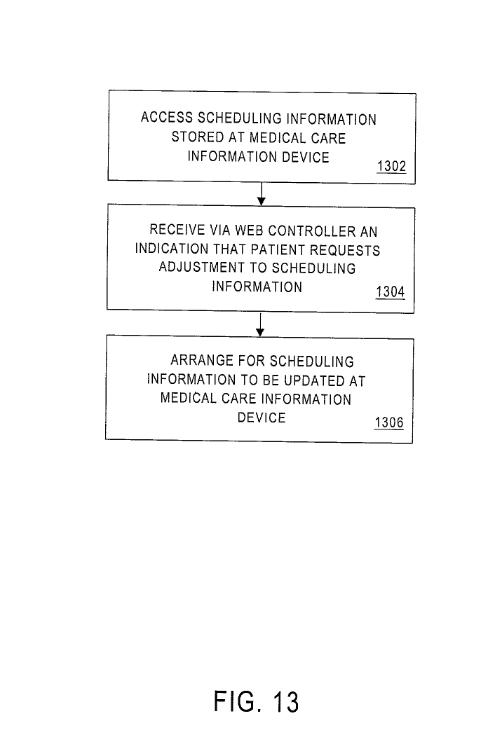

Methods that may be used in connection with the medical care information system 100 according to some embodiments of the present invention will now be described in detail with respect to FIGS. 12 and 13.

Medical Care Information System Methods

FIG. 12 is a flow chart of a computer-implemented method for facilitating an exchange of information at a controller 800. At 1202, treatment scheduling information associated with a patient is retrieved from a medical care information database. For example, the controller 800 may transfer treatment scheduling information from a LANTIS® Oncology Management System database into a local scheduling database 1000 when a patient accesses a Web site.

At 1204, an image of the patient is retrieved. For example, the controller 800 may retrieve an image of the patient from a local patient database 900 or from a LANTIS® Oncology Management System database.

The treatment scheduling information and the image of the patient are transmitted to the patient via a Web site at 1206. For example, the controller 800 may transmit the scheduling information and the image of the patient via a patient display as illustrated in FIG. 3.

If no indication associated with the treatment scheduling information (e.g., a request to adjust the schedule) is received from the patient via the Web site at 1208, the process ends at 1210.

If an indication is received at 1208, the indication is converted into an electronic mail message at 1212. For example, the controller 800 may convert HTML information into an electronic mail message (e.g., indicating the patient and the requested adjustment to his or her schedule). The electronic mail message is the transmitted at 1214 (e.g., to a medical care information device 110 and/or a medical care provider device 120).

FIG. 13 is a flow chart of a method that may be performed by a medical care provider device 120 according to an embodiment of the present invention. At 1302, scheduling information stored at the medical care information device 110 is accessed. For example, a medical care provider (e.g., a doctor or nurse) may use a medical care provider device 120 (e.g., a PC or work station) to access scheduling information stored via a LANTIS® Oncology Management System. In particular, the medical care provider may input and retrieve information associated with a patient's medical care appointments.

At 1304, an indication that a patient requests an adjustment to scheduling information is received via the controller 800 (e.g., via a Web controller associated with a Web site accessed by the patient). For example, the controller 800 may arrange for the indication to be displayed via the medical care provider device 120 (e.g., by arranging for an electronic mail message to be displayed via the medical care provider device 120).

It is then arranged for the scheduling information to be updated at the medical care information device 110 at 1306. For example, the medical care provider may use the medical care provider device 120 to update the information stored via a LANTIS® Oncology Management System. According to another embodiment, the medical care provider simply indicates whether or not the patient's requested adjustment to the scheduling information is acceptable, and the information stored at the medical care information device 110 is automatically updated accordingly.

In this way, an exchange of information between a patient and a medical care provider is facilitated. In particular, information may be provided to the patient without the medical care provider manually providing, or the patient manually transferring, the information. As a result, the exchange may be more convenient for the patient (and for the medical care provider) and inadvertent errors may be reduced. Moreover, the confidential nature of the information stored at the medical care information device 110 may be protected.

Additional Embodiments

The following illustrates various additional embodiments of the present invention. These do not constitute a definition of all possible embodiments, and those skilled in the art will understand that the present invention is applicable to many other embodiments. Further, although the following embodiments are briefly described for clarity, those skilled in the art will understand how to make any changes, if necessary, to the above-described apparatus and methods to accommodate these and other embodiments and applications.

Although many embodiments have been described with respect an exchange of information between a patient and a medical care provider, the present invention may also be used to exchange information between various medical care providers. For example, the controller 800 may arrange for scheduling information to be transmitted from a first medical care provider to a second medical care provider.

According to another embodiment, the patient device 400 sends an indication (e.g., a request to adjust scheduling information) directly to the medical care provider device 120 (e.g., instead of sending the indication via the controller 800). For example, a patient may use a Web site associated with a controller 800 to communicate directly with a medical care provider device 120. Similarly, the medical care provider device 120 may send information (e.g., an adjustment to scheduling information) directly to the patient device 400.

According to still another embodiment, a medical care provider may enter information into the medical care provider device 120 for the purpose of being displayed to a patient. In this case, the information may be stored at the medical care information device 110 (e.g., via a LANTIS® Oncology Management System) and forwarded to the patient device 400 via the controller 800 when the patient accesses a Web site.

According to another embodiment, the controller 800, the medical care information device 110, and/or the medical care provider device 120 may be co-located and/or incorporated into a single device. For example, all of these devices may be located at a hospital or medical clinic.

According to another embodiment, the controller 800 communicates with both the patient device 400 and the medical care information device 110 via the same communication network 10. For example, the controller 800 may communicate with both the patient device 400 and the medical care information device 110 via the Internet. In this case, encryption techniques may be used to protect the confidential nature of the patient's medical care information. Moreover, a number of different medical care provider devices 120 may be associated with a medical care provider. For example, a first medical care provider device may be used to enter information into the medical care information device 110 and second medical care provider device may be used to receive information from a patient.

The present invention has been described in terms of several embodiments solely for the purpose of illustration. Persons skilled in the art will recognize from this description that the invention is not limited to the embodiments described, but may be practiced with modifications and alterations limited only by the spirit and scope of the appended claims.

What is claimed is:

1. A method for facilitating an exchange of information associated with medical care provided to a patient by a medical care provider, comprising:
exchanging medical care information with a medical care information device, the medical care information device being in communication with a medical care provider device associated with the medical care provider; and
exchanging patient information with a remote patient device via a communication network, the patient information being associated with the medical care information.

2. The method of claim 1, wherein said exchanging medical care information comprises retrieving scheduling information from the medical care information device, and said exchanging patient information comprises transmitting the scheduling information to the remote patient device.

3. The method of claim 2, further comprising:
receiving from the remote patient device an indication associated with the scheduling information; and
arranging for the indication to be transmitted to the medical care provider device.

4. The method of claim 3, wherein the indication comprises at least one of: (i) a request to adjust the scheduling information; (ii) an adjustment to the scheduling information, and (iii) a confirmation of the scheduling information.

5. The method of claim 3, wherein said receiving comprises receiving the indication via a Web page and said arranging comprises converting the indication to an electronic mail message.

6. The method of claim 3, wherein said arranging comprises at least one of: (i) transmitting the indication to the medical care provider device, and (ii) storing the indication via the medical care information device.

7. The method of claim 2, further comprising:
retrieving supplemental information associated with the patient; and
transmitting the supplemental information to the remote patient device.

8. The method of claim 7, wherein the supplemental information comprises at least one of: (i) a patient name, (ii) an image of the patient, and (iii) information associated with a medical care provider.

9. The method claim 2, further comprising:
determining at least one update to the scheduling information; and
transmitting an indication of the update to the remote patient device.

10. The method of claim 2, wherein the scheduling information includes at least one of: (i) the medical care provider, (ii) a medical care treatment, (iii) a medical care category, (iv) an appointment date, (v) an appointment time, and (vi) an appointment duration.

11. The method of claim 2, wherein the scheduling information is associated with at least one of: (i) a medical examination, (ii) a medical treatment, and (iii) a medical procedure.

12. The method of claim 2, wherein the scheduling information is associated with a plurality of at least one of: (i) medical care providers, and (ii) medical care information devices.

13. The method of claim 1, wherein said exchanging patient information comprises receiving journal information from the remote patient device, and said exchanging medical care information comprises transmitting a least a portion of the journal information to the medical care information device.

14. The method of claim 13, wherein the journal information comprises at least one of: (i) text information, (ii) audio information, and (iii) image information.

15. The method of claim 13, further comprising:
receiving from the remote patient device an indication of the portion of the journal information that should be transmitted to the medical care information device.

16. The method of claim 1, wherein said exchanging medical care information comprises retrieving medical diagnosis information from the medical care information device, and said exchanging patient information comprises transmitting to the remote patient device at least one of: (i) information associated with another patient, and (ii) information associated with a support group.

17. The method of claim 16, further comprising:
determining address information associated with the patient, wherein the patient information transmitted to the remote patient device is further based on the address information.

18. The method of claim 1, wherein said exchanging medical care information comprises retrieving medical diagnosis information from the medical care information device, and said exchanging patient information comprises transmitting educational information to the remote patient device.

19. The method of claim 18, wherein the educational information comprises at least one of: (i) information associated with the medical care provider, (ii) information generated by the medical care provider, and (iii) information reviewed by the medical care provider.

20. The method of claim 1, wherein said exchanging medical care information comprises retrieving at least one medical instruction from the medical care information device and said exchanging patient information comprises transmitting the medical instruction to the remote patient device.

21. The method of claim 20, further comprising:
receiving from the remote patient device an indication that the patient has complied with the medical instruction.

22. The method of claim 1, wherein the medical care provider device is associated with at least one of the following: (i) the medical care provider, (ii) a hospital, (iii) a medical clinic, and (iv) a doctor.

23. The method of claim 1, wherein the medical care information device is adapted to exchange at least one of the following with the medical care provider device: (i) scheduling information, (ii) clinical data, (iii) an electronic medical record associated with the patient, (iv) budget information, (v) oncology information, (vi) chemotherapy charting, (vii) outcome management, (viii) clinical imaging, and (ix) transcription information.

24. The method of claim 1, wherein the remote patient device comprises at least one of: (i) a personal computer, (ii) a portable computing device, (iii) a telephone, (iv) an interactive television device, and (v) a kiosk.

25. The method of claim 1, wherein the communication network comprises at least one of: (i) the Internet, (ii) a public network, (iii) a public switched telephone network, (iv) a proprietary network, (v) a wireless network, and (vi) a local area network.

26. A controller, comprising:
a processor; and
a storage device in communication with said processor and storing instructions adapted to be executed by said processor to:
exchange medical care information with a medical care information device, the medical care information device being in communication with a medical care provider device associated with a medical care provider; and
exchanging patient information with a remote patient device via a communication network, the patient information being associated with the medical care information.

27. The controller of claim 26, wherein said storage device further stores at least one of: (i) a patient database, (ii) a scheduling database, and (iii) a supplemental information database.

28. The controller of claim 26, further comprising:
a communication device coupled to said processor and adapted to communicate with at least one of: (i) a patient device, (ii) a medical care information device, and (iii) a medical care provider device.

29. A medium storing instructions adapted to be executed by a processor to perform a method of facilitating an exchange of information associated with medical care provided to a patient by a medical care provider, said method comprising:
exchanging medical care information with a medical care information device, the medical care information device being in communication with a medical care provider device associated with the medical care provider; and
exchanging patient information with a remote patient device via a communication network, the patient information being associated with the medical care information.

30. A computer-implemented method for facilitating an exchange of information associated with medical care provided to a patient by a medical care provider, comprising:
retrieving treatment scheduling information associated with the patient from a medical care information database, the medical care information database also being accessed by a medical care provider device associated with the medical care provider;
retrieving an image of the patient;
transmitting to the patient the treatment scheduling information and the image of the patient via a Web site;
receiving from the patient an indication associated with the treatment scheduling information via the Web site;
converting the indication into an electronic mail message; and
arranging for the electronic mail message to be transmitted to the medical care provider device.

31. A method for facilitating an exchange of information associated with medical care provided to a patient by a medical care provider, comprising:
exchanging patient information at a patient device via a communication network, the patient information being based on medical care information stored at a medical care information device accessed by the medical care provider; and
transmitting from the patient device an indication associated with the patient information.

32. A method for facilitating an exchange of information associated with medical care provided to a patient by a medical care provider, comprising:
receiving scheduling information at a patient device via a communication network, the scheduling information being based on information stored at a medical care information device accessed by the medical care provider; and
transmitting from the patient device a request to adjust the scheduling information.

33. A method for facilitating an exchange of information associated with medical care provided to a patient by a medical care provider, comprising:
exchanging medical care information with a medical care information device, the medical care information device being in communication with a controller that that transmits patient information to a remote patient device based on the medical care information; and
receiving an indication associated with the patient information.

34. A method for facilitating an exchange of information associated with medical care provided to a patient, comprising:
exchanging medical care information with a medical care information device via a proprietary communication network; and
exchanging patient information with a remote patient device via a public communication network, the patient information being associated with the medical care information.
